(19) United States
(12) Patent Application Publication
(10) Pub. No.: US 2008/0300421 A1
(43) Pub. Date: Dec. 4, 2008

(54) Process for purifying theanine

(75) Inventor: Stanislas Xavier Baudouin, Aubergenville (FR)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco,Inc. d/b/a Unilever

(21) Appl. No.: 12/151,621

(22) Filed: May 8, 2008

(30) Foreign Application Priority Data
May 11, 2007 (EP) EP07108061

(51) Int. Cl. C07C 227/40

(52) U.S. Cl. 562/554

(57) ABSTRACT

The present invention provides a process for purifying theanine from an aqueous solution having a dominant cation and comprising impurities. The process comprises the steps of: introducing the aqueous solution into a column in an amount of from 2 to 20% of the column volume, wherein the column is packed with a cation exchange resin of the same cation type as the dominant cation; then introducing an aqueous mobile phase having a pH of from 2.5 to 8.5 into the column thereby to elute a zone enriched in the impurities followed by a zone enriched in theanine; and then recovering at least one fraction of the zone enriched in theanine.

### TECHNICAL FIELD OF THE INVENTION

The present invention relates to a process for purifying the amino acid theanine.

### BACKGROUND TO THE INVENTION

Food and beverage products which can enhance mental and physical aspects of the human body are becoming increasingly popular. In particular, products which produce an enhanced state of mental acuity are especially commercially valuable.

Theanine is an amino acid which, within the plant kingdom, is uniquely found in tea (Camellia sinensis). Theanine has been found to have numerous beneficial effects on the human body and mind. For example, it is reported that theanine stimulates α-waves in the mammalian brain and bestows a relaxed but alert mental state to the individual. These physiological effects are particularly apparent at specific dosage levels. For example, International Patent Application no. PCT/EP2005/012464 (Unilever PLC et al.) describes consumable compositions which comprise specific levels of theanine and caffeine and which are shown to provide noticeable improvements in concentration, mental focus and/or alertness of an individual consuming the compositions.

Although tea is relatively rich in theanine, in fact theanine only comprises about 2% by weight of the extractable tea solids in tea plant material. Thus, synthetic theanine (e.g. Suntheanine™ produced by Taiyo Kagaku) has been developed to meet the increasing demand for products with enhanced levels of theanine.

The modern consumer is, however, particularly interested in naturally healthy beverages which form part of a modern healthy lifestyle. Tea fits well with this attitude in view of its natural content of inter alia antioxidants. There is therefore a need to provide methods for the concentration of naturally occurring theanine without adding synthetic compounds.

In an attempt to meet this need, processes have been developed which use membrane filtration to concentrate theanine in tea extracts. For example, international patent application no. PCT/EP2005/010376 discloses a process to provide a theanine-rich tea extract comprising the steps of performing a cold water extraction of tea plant material and then passing the extract through a nanofiltration step. The process results in a permeate having tea solids enriched in theanine.

We have found, however, that even such permeates may contain a significant amount of impurities. In particular, impurities such as saccharides and amino acids have proved difficult to remove from the permeates owing to their physico-chemical similarity with theanine. Furthermore, the presence of some impurities makes conventional drying (e.g. spray drying) of the permeate particularly problematic.

US patent application published as US 2005/0084544 (Procter & Gamble Co. Inc.) describes a process where a tea extract is contacted with a material which adsorbs the polyphenolic materials. This process generates a theanine-rich extract substantially free of polyphenols. However, in this case too the extract still contains many impurities which require a further filtration step to be removed.

U.S. Pat. No. 5,922,380 (Ito En Ltd.) discloses a process where a tea extract is contacted with a cation exchange resin ionically bonded to potassium ions. The aim of the process is to remove divalent metal ions from the tea extract whilst retaining other components that contribute to tea flavour. Thus the process involves flowing the extract through a column packed with the resin under conditions where the only separation mechanism is cation exchange. Consequently, the process of U.S. Pat. No. 5,922,380 does not result in separation of theanine from any impurities other than multivalent metal ions.

Thus we have recognised that there is a need to provide a simple process for purifying theanine from a mixture containing impurities, including some neutral and/or anionic impurities. We have also recognised that there is a need to provide such a process which does not require the use of synthetic chemicals and/or organic solvents.

### DEFINITIONS

### Impurities

As used herein, the term “impurities” refers to substances other than theanine or water.

### Dominant Cation

The dominant cation in a solution is that cation present in the solution at a higher molar concentration than any other cation in the solution.

### Zone Enriched in Impurities

A zone enriched in impurities is a chromatographic zone wherein the concentration of impurities (expressed as percent of the total solids in the zone) is greater than the concentration of impurities in the aqueous solution (expressed as percent of the total solids in the aqueous solution) at the start of the process.

### Zone Enriched in Theanine

A zone enriched in theanine is a chromatographic zone wherein the concentration of theanine (expressed as percent of the total solids in the zone) is greater than the concentration of theanine in the aqueous solution (expressed as percent of the total solids in the aqueous solution) at the start of the process.

### Tea Extract

As used herein, the term “tea extract” refers to dry material from the leaves and/or stem of Camellia sinensis var. sinensis and/or Camellia sinensis var. assamica. The leaves and/or stem may have been subjected to a so-called “fermentation” step wherein they are oxidised by certain endogenous enzymes that are released during the early stages of “black tea” manufacture. This oxidation may even be supplemented by the action of exogenous enzymes such as oxidases, laccases and peroxidases. Alternatively the leaves may have been partially fermented (“oolong” tea) or substantially unfermented (“green tea”).

### Chromatographic Terms

The terms “mobile phase”, “column”, “column volume”, “elute” and “zone” are standard chromatographic terms as defined in the “Compendium of Analytical Nomenclature: Definitive Rules 1997”, IUPAC, 3^{rd} Edition, Chapter 9.

### SUMMARY OF THE INVENTION

The present invention provides a process for purifying theanine from an aqueous solution having a dominant cation and comprising impurities, the process comprising the steps of:
a. introducing the aqueous solution into a column in an amount (Vₐ) of from 2 to 20% of the column volume (V_{c}), wherein the column is packed with a cation exchange resin of the same cation type as the dominant cation; then
b. introducing an aqueous mobile phase having a pH of from 2.5 to 8.5 into the column thereby to elute a zone enriched in the impurities followed by a zone enriched in theanine; then
c. recovering at least one fraction of the zone enriched in theanine; and then
d. optionally concentrating and/or drying the at least one fraction of the zone enriched in theanine.

Operation of the process as specified ensures that the dominant separation mechanism is that of ion exclusion and results in separation of the theanine and impurities into substantially distinct zones.

### DETAILED DESCRIPTION

The aqueous solution from which the theanine is purified contains impurities. The impurities may comprise cations, anions, neutral molecules or a mixture thereof. Typically the impurities will comprise saccharides, amino acids or a mixture thereof. The saccharides may be monosaccharides, disaccharides, oligosaccharides, polysaccharides or a mixture thereof. The impurities may additionally or alternatively comprise polyphenols, caffeine or a mixture thereof.

The theanine is preferably from a natural source. The most preferred source of theanine is tea. Thus in a preferred embodiment the aqueous solution is an aqueous tea extract. It is particularly preferred that the tea extract is a black tea extract. The aqueous tea extract may be provided directly by extraction of tea leaves with water. Alternatively, the aqueous tea extract may have been subjected to one or more purification and/or concentration steps following extraction. For example, the tea extract may have been subjected to nanofiltration using the process of PCT/EP2005/010376 which is hereby incorporated by reference.

The aqueous solution will contain one or more cations. In most cases (and especially if the aqueous solution is an aqueous tea extract) the dominant cation is potassium (K⁺).

It is preferred that the aqueous solution is substantially free from multivalent cations. Multivalent cations are difficult to remove from the resin and so foul the column, resulting in short resin lifetimes. Thus it is preferred that the aqueous solution comprises greater than 80% monovalent cations by weight of the cations in the solution, more preferably greater than 90% and most preferably from 95 to 99.99%.

Because the aqueous solution is introduced to the column in a relatively small amount, it is preferable for the solution to have a high solids content in order to maximise the efficiency of the separation and to minimise the amount of water to be removed if the fraction is dried after step c. Thus it is preferred that the aqueous solution has a total solids content of at least 2% by weight of the solution, more preferably at least 5%, most preferably at least 10%. The solids content should not be too high, however, otherwise the sample becomes too viscous and/or problems may arise with insoluble material developing during the separation. Therefore it is preferred that the solids content is less than 50% by weight of the solution, more preferably less than 40% and most preferably less than 30%.

To maximise the yield of theanine it is preferred that the total solids comprise theanine in an amount of at least 1% by weight of the solids, more preferably at least 2%, more preferably still at least 4%, and most preferably at least 6%. Typically the total solids will comprise less than 80% theanine by weight of the solids. The process is particularly suited for purifying theanine from aqueous solutions wherein the total solids comprise less than 60% theanine by weight of the total solids, or even less than 40%.

To ensure that the mechanism of separation is that of ion exclusion, the theanine is eluted at a pH close to its pI. It is therefore preferred that the pH of the aqueous solution is in the range 2.5 to 8.5, more preferably in the range 3.5 to 7.5, more preferably still in the range 4 to 7, and most preferably from 5 to 6. It is especially important that the pH of the solution is within these ranges if the mobile phase is not buffered.

The aqueous solution is introduced into the column in an amount (Vₐ) of from 2 to 20% of the column volume (V_{c}). This ensures that the aqueous solution only occupies a small proportion of the column and thus allows for separation of the theanine and impurities when eluted. The amount should not be too small, however, otherwise the efficiency of the process is compromised. Preferably Vₐ is from 3 to 15% of V_{c}, more preferably from 5 to 10%.

The column is packed with a cation exchange resin of the same cation type as the dominant cation. Usually this will mean that the resin is in the potassium (K⁺) form.

Suitable resins include those having functional groups for cation exchange such as a sulfonic acid group (—SO₃M), a phosphoric acid group (—PO(OM)₂), or a carboxyl group (—COOM); wherein M is the dominant cation. A resin having a sulfonic acid group, such as sulfonated polystyrenic resins, are particularly preferred. Such resins include styrene-divinylbenzene copolymer resins. Suitable commercially available resins include the Diaion™ UBK range including UBK530, UBK535, UBK550 and UBK555 (made by Mitsubishi Kagaku Co.).

Once the aqueous solution has been introduced into the column an aqueous mobile phase is then introduced to elute the solution. In order that the theanine is separated from the impurities it is necessary that the mobile phase has a composition different to that of the aqueous solution. In particular the mobile phase should be substantially free of theanine. Preferably the mobile phase should comprise less than 0.01% theanine by weight of the mobile phase, more preferably less than 0.001% and optimally less than 0.0001%.

The aqueous mobile phase has a pH of from 2.5 to 8.5 in order that theanine is in the neutral form. Preferably the pH of the mobile phase is from 3.5 to 7.5, more preferably from 4 to 7, and most preferably from 5 to 6. If the aqueous solution is strongly acidic or basic (e.g. has a pH outside of the range of 2.5 to 8.5) then it may be necessary that the mobile phase is buffered. In this case the mobile phase will comprise buffer salts. For example the mobile phase may comprise a phosphate buffer.

It is preferred, however, to avoid the use of any chemical additives. Thus in a preferred embodiment the mobile phase is substantially pure water. For example, the mobile phase may comprise less than 1000 ppm total solids, more preferably less than 500 ppm, and most preferably from 1 ppb to 100 ppm. In a particularly preferred embodiment the mobile phase is deionised water.

It is also preferred that the mobile phase is substantially free from multivalent cations. Multivalent cations are difficult to remove from the resin and so foul the column, resulting in short resin lifetimes. Thus it is preferred that the mobile phase comprises greater than 80% monovalent cations by weight of the cations in the mobile phase, more preferably greater than 90% and most preferably from 95 to 99.99%.

In order to avoid the formation of air bubbles in the column which may interfere with the separation it is preferred that the mobile phase is degassed prior to introduction to the column. Any known means for degassing may be employed including sonication or sparging with a water-insoluble gas such as nitrogen.

As the mobile phase passes down the column, the neutral theanine is retarded and/or weakly adsorbed by the resin whilst other molecules of different size and/or charge are excluded from the resin. As a result, a zone enriched in the impurities is formed in advance of a zone enriched in theanine.

It is preferred that the zones are eluted at a high temperature to reduce the effect of the viscosity of the relatively concentrated zones on the flow rate. Typically the temperature will be in the range of 45 to 90° C., more preferably 55 to 80° C.

The use of high temperatures also reduces the risk of microbiological contamination of the column.

The next step of the process involves recovering at least one fraction of the zone enriched in theanine. Typically this will be achieved by collecting fractions of the eluate exiting from the column and retaining those fractions comprising a significant amount of theanine. Where multiple fractions are collected, they may be pooled.

Fractions of the zone enriched in impurities are typically discarded. However, if this zone is enriched in valuable impurities, such as polyphenols, then the process may comprise the additional step of recovering at least one fraction of the zone enriched in impurities.

The at least one fraction of the zone enriched in theanine recovered in step c may be used directly without further processing. For example it may be added directly to a food or beverage product. In particular, owing to the high efficiency of separation there is generally no need to further purify the fraction, e.g. with a membrane-filtration step, prior to use and/or drying.

In a preferred embodiment the at least one fraction of the zone enriched in theanine is concentrated and/or dried. This allows for stable long-term storage of the fraction. Typically the fraction will be dried to less than 20% moisture by weight, more preferably less than 10% and optimally to 1 to 7% moisture.

### EXAMPLES

### Example 1

This example demonstrates purification of theanine from a concentrated aqueous tea extract.

### Preparation of the Aqueous Tea Extract:

Broken mixed fannings (BMF) of black tea were extracted with water. The resulting extract was centrifuged, nanofiltered and dried to yield a tea extract comprising 6 wt % theanine, 16 wt % mono- and di-saccharides, 15 wt % ash, 6 wt % polyphenols, 6 wt % amino acids other than theanine, and 4 wt % caffeine.

The tea extract was then re-dissolved in water and filtered through a 5 μm filter to yield the aqueous tea extract having a total solids content of 20 wt % and pH of 4.85.

### Preparation of the Column:

The resin used was Diaion™ UBK550 in the K⁺ form. This resin is a sulfonated polystyrenic polymer in the form of beads with a narrow size distribution and an average diameter of 220 μm.

The column was half-filled with degassed deionised water. Resin was then added and hot water recycled through the column for 30 minutes to adjust the resin level.

The column was then packed. This was achieved by performing two elutions without any sampling or data recording, using a flow rate of 4 V_{c} per hour. This resulted in compaction due to the swelling and the shrinking of the resin, following injection of product then water. After these two elutions, the resin level was adjusted to the top of the column by adjusting a piston in the column.

The packed column had a height of 113 cm and a diameter of 1.5 cm. Thus the column volume (V_{c}) was 200 ml. The column was fitted with a water jacket and maintained at a temperature of 60° C.

### Separation of the Extract:

The tea extract was injected into the column in an amount of 9 ml (0.05 V_{c}). The mobile phase (degassed deionised water) was then pumped through the column at a rate of 5 ml per minute (1.5 V_{c} per hour). The output from the column was fed to a fraction collector with a fresh fraction being collected every minute (equivalent to every 0.025 V_{c} of product eluted). Each fraction was analysed for total solids (Brix scale using a Bellingham & Stanley RFM 3000 refractometer) and theanine content (using High Performance Liquid Chromatography). The results are shown in table 1.

TABLE 1

| | | Total Solids | Theanine | Theanine |
|---|---|---|---|---|
| | Volume | Concentration | Concentration | Concentration in |
| Fraction | Eluted | in Fraction | in Fraction | Total Solids |
| No. | (% of V_{c}) | (wt %) | (g/l) | (wt %) |
| 1 | 29.3 | 0.1 | 0.00 | 0.00 |
| 2 | 31.8 | 0.1 | 0.00 | 0.00 |
| 3 | 34.3 | 0.1 | 0.00 | 0.00 |
| 4 | 36.8 | 0.5 | 0.00 | 0.00 |
| 5 | 39.3 | 1.5 | 0.00 | 0.00 |
| 6 | 41.8 | 3.4 | 0.00 | 0.00 |
| 7 | 44.3 | 5.4 | 0.00 | 0.00 |
| 8 | 46.8 | 5.4 | 0.00 | 0.00 |
| 9 | 49.3 | 3.0 | 0.00 | 0.00 |
| 10 | 51.8 | 1.9 | 0.00 | 0.00 |
| 11 | 54.3 | 1.8 | 0.00 | 0.00 |
| 12 | 56.8 | 1.9 | 0.29 | 1.53 |
| 13 | 59.3 | 1.9 | 0.75 | 3.92 |
| 14 | 61.8 | 2.0 | 1.37 | 6.81 |
| 15 | 64.3 | 2.0 | 1.78 | 8.85 |
| 16 | 66.8 | 1.9 | 2.63 | 13.76 |
| 17 | 69.3 | 1.5 | 2.61 | 17.32 |
| 18 | 71.8 | 1.1 | 2.30 | 20.78 |
| 19 | 74.3 | 0.8 | 1.95 | 24.34 |
| 20 | 76.8 | 0.6 | 1.53 | 25.48 |
| 21 | 79.3 | 0.4 | 1.09 | 27.13 |
| 22 | 81.8 | 0.3 | 0.79 | 26.38 |
| 23 | 84.3 | 0.2 | 0.65 | 32.59 |
| 24 | 86.8 | 0.2 | 0.54 | 26.91 |
| 25 | 89.3 | 0.1 | 0.41 | 40.68 |
| 26 | 91.8 | 0.1 | 0.44 | 43.82 |
| 27 | 94.3 | 0.1 | 0.40 | 40.30 |

As is apparent from the data in table 1, most of the impurities are eluted in the first 60% of the column volume whilst theanine is only beginning to be eluted at this point. Thus fractions collected after this point (fractions 14 onwards) are enriched in theanine compared with the aqueous tea extract which was injected into the column (theanine content of 6 wt % on dry matter).

1. A process for purifying theanine from an aqueous solution having a dominant cation and comprising impurities, the process comprising the steps of:
a. introducing the aqueous solution into a column in an amount (Vₐ) of from 2 to 20% of the column volume (V_{c}), wherein the column is packed with a cation exchange resin of the same cation type as the dominant cation; then
b. introducing an aqueous mobile phase having a pH of from 2.5 to 8.5 into the column thereby to elute a zone enriched in the impurities followed by a zone enriched in theanine; and then
c. recovering at least one fraction of the zone enriched in theanine.

2. A process according to claim 1 wherein the aqueous solution has a total solids content of at least 2% by weight of the solution, preferably at least 5%, more preferably at least 10%.

3. A process according to claim 2 wherein the total solids comprise theanine in an amount of at least 1% by weight of the solids.

4. A process according to claim 1 wherein the impurities comprise saccharides, amino acids or a mixture thereof.

5. A process according to claim 1 wherein the pH of the mobile phase is from 4 to 7.

6. A process according to claim 1 wherein Vₐ is from 3 to 15% of V_{c}.

7. A process according to claim 1 wherein the mobile phase is deionised water.

8. A process according to claim 1 wherein the dominant cation is potassium (K⁺).

9. A process according to claim 1 wherein the aqueous solution is an aqueous tea extract.

10. A process according to claim 1 comprising the additional step of:
d. Concentrating and/or drying the at least one fraction of the zone enriched in theanine.

11. A process according to claim 10 wherein the at least one fraction is not subjected to membrane-filtration between steps c and d.

12. A process according to claim 1 wherein the resin is a sulfonated polystyrenic resin.
